(19) United States
(12) Patent Application Publication
(10) Pub. No.: US 2026/0287550 A1
(43) Pub. Date: Sep. 24, 2026

(54) CARBOHYDRATE-BASED TRIBOELECTRIC SENSORS AND DEVICES INCLUDING FOR FOOD QUALITY MONITORING

(71) Applicant: THE BOARD OF TRUSTEES OF THE UNIVERSITY OF ILLINOIS, Urbana, IL (US)

(72) Inventors: Yi-Cheng Wang, Champaign, IL (US); Zhenhui Jin, Wuxi (CN); Yang Fu, Champaign, IL (US)

(73) Assignee: THE BOARD OF TRUSTEES OF THE UNIVERSITY OF ILLINOIS, Urbana, IL (US)

(21) Appl. No.: 19/573,491

(22) Filed: Mar. 20, 2026

Related U.S. Application Data
(60) Provisional application No. 63/775,058, filed on Mar. 20, 2025.

(51) Int. Cl. G01N 27/60; G01N 33/02

(52) U.S. Cl. CPC G01N 27/60; G01N 33/02

(57) ABSTRACT

Aspects of the subject disclosure may include, for example, a triboelectric device comprising a device body including a carbohydrate polymer, and a plasticizer, where the carbohydrate polymer includes pectin and the plasticizer includes glycerol, where the concentration of glycerol is approximately 50% by weight relative to the pectin if the triboelectric device is being used as a sensor for food quality monitoring or is approximately 70% by weight relative to the pectin if the triboelectric device is being used as an energy harvester, where when mechanical energy is applied to the device body electrical energy is generated.

### CROSS-REFERENCE TO RELATED APPLICATION

This application claims the benefit of and priority to U.S. Provisional Ser. No. 63/775,058, filed Mar. 20, 2025. The contents of the foregoing are hereby incorporated by reference into this application as if set forth herein in full.

### GOVERNMENT SUPPORT

This invention was made with government support under NI23HFPXXXXXG033, NI22HFPXXXXXG030, NI21HFPXXXXXG009, NI20HFPXXXXXG050, 2019-38502-30120, and 2020-38502-32916 awarded by the US Department of Agriculture National Institute of Food and Agriculture. The government has certain rights in the invention.

### FIELD OF THE DISCLOSURE

The subject disclosure generally relates to energy harvesting and self-powered sensing devices utilizing carbohydrate-based materials, such as for triboelectric sensors, devices or nanogenerators or generators. This can include sensors for food quality monitoring.

### BACKGROUND

Energy sources, particularly utilizing clean energy, are often products that are sought after throughout the world.

Humidity can critically affect the quality of food. Low-moisture food is a class of products with low water activity, which either occur naturally or are made by drying or dehydrating higher moisture foods. Examples include dried fruits, nuts, cereals, powdered milk, and certain snack foods. Due to their low water activities, such foods are relatively less susceptible to the growth of foodborne pathogens and microbial spoilage. However, research has shown that relative humidity (RH) can significantly affect the quality of these foods. Badii et al. (Effect of Storage Relative Humidity on Physical Stability of Dried Fig, Journal of Food Processing and Preservation 38 (2014) 477-483) for example, reported that storage of dried figs at high RH levels could result in undesired physical properties, as variations in water content affected their glass-transition temperatures (Tg) and led to crystallization of amorphous sugars in their structures. Alongside these and other negative effects on their textures and appearances, various studies have shown that low-moisture foods' flavors and nutrient qualities can also be damaged if RH is not monitored and controlled properly during storage. Therefore, monitoring the RH levels of low-moisture food can be very important.

To better monitor RH change during storage, various humidity sensors have been developed based on colorimetry or on measurement of variation in electrical properties such as dielectric permittivity and capacitance, among other mechanisms. However, these types of monitoring have yet been widely applied due to various limitations, such as toxicity concerns, in the case of some colorimetric humidity sensors; the others' electricity requirements; and cost issues. Therefore, it is important to develop safe, easy-to-use, low-cost sensors that can provide reliable RH information to low moisture food consumers and other stakeholders.

### BRIEF DESCRIPTION OF THE DRAWINGS

Reference will now be made to the accompanying drawings, which are not necessarily drawn to scale, and wherein:

FIG. 1. (a) Synthesis process of pectin-glycerol (PG) films. (b) Schematic and (c) mechanism illustration of glycerol's plasticizing effect within PG films. Digital pictures of (d) a piece of PG film without the addition of glycerol (PG-0 film) and (e) a piece of PG film containing glycerol weighing 50% as much as the pectin (PG-50 film). (f-g) Atomic-force microscope images of the surfaces of (f) the PG-0 film and (g) the PG-50 film.

FIG. 2. (a) Schematic of the assembly of a pectin-glycerol film (PG film)-based triboelectric nanogenerator (P-TENG) on a linear motor. (b) Schematic of an electron-cloud potential-well model of the contact electrification between PG-film and polytetrafluoroethylene (PTFE). (c) Schematic of the working mechanism of the P-TENG. (d) Open-circuit voltage (Voc), (e) short circuit current (Isc), and (f) short-circuit charge (Qsc) outputs of a P-TENG working at frequencies from 1 Hz to 5 Hz in a controlled relative humidity of 55%.

FIG. 3. Variations in the amplitudes of (a) voltage and current and (b) power density across a series of resistors connected to a pectin-glycerol film-based triboelectric nanogenerator (PTENG). (c) The P-TENG successfully lighting up 27 light-emitting diodes (LEDs) connected in series. The P-TENG was first connected to a full-bridge rectifier, which was further connected to the LEDs. (d) Schematic of a capacitor storing the electrical output of the P-TENG connected to a full-bridge rectifier. (e) Charging curves of different capacitors connected to the P-TENG that was connected to a full-bridge rectifier. (f) An electronic calculator (left) and a hygrometer (right) were successfully powered by the P-TENG when connected to a full-bridge rectifier and a capacitor, respectively.

FIG. 4. (a) Molecular model of galacturonic acid. (b) Formation of hydrogen bonds between water molecules and a galacturonic-acid molecule. (c-d) Schematic of the water-sorption behaviors of pectin-glycerol (PG) films at (c) lower and (d) higher relative-humidity levels. (e) Variation in the dry-basis (d.b.) moisture content of PG-50 films—i.e., PG films whose glycerol content weighs 50% as much as their pectin content—when stored in 33%, 55%, 75% and 99% relative humidity. (f) Fourier-transform infrared spectroscopic analysis of PG-50 films stored at 16%, 33%, 55%, 75% and 99% relative humidity.

FIG. 5. (a) Voltage output of pectin-glycerol film-based triboelectric nanogenerators (PTENGs) in which one of the triboelectric layers is PG-50 film, i.e., pectin-glycerol film whose glycerol content weighs 50% as much as its pectin content. Measurements were performed after P-TENGs were stored at five different relative-humidity (RH) levels for 3 days. Different letters above each column indicate significant differences (p<0.05). (b-d) Illustrations of the output variations of P-TENG stored at (b) low, (c) intermediate and (d) high RH levels.

FIG. 6. (a) Voltage output of pectin-glycerol film-based triboelectric nanogenerators (PTENGs) in which one of the triboelectric layers is PG-50 film, i.e., pectin-glycerol film whose glycerol content weighs 50% as much as its pectin content, after water-sorption and water desorption processes under different relative-humidity conditions. Asterisks indicate that at a given relative-humidity level, there was a significant difference between the voltage outputs of the devices that underwent the water-sorption and water-desorption processes (p<0.05). (b) Working mechanism of a P-TENG used as a humidity sensor. (c) Variations in crackers' quality when they were stored at low and high relative-humidity levels. (d) Variations in crackers' dry basis (d.b.) moisture content when stored at different relative-humidity levels. Black: water sorption process; red: water-desorption process. (e) Variations in crackers' hardness when stored in different relative-humidity levels. Purple: water-sorption process; blue: water desorption process.

FIG. 7. (a) Structural schematic of a pectin-glycerol triboelectric nanogenerator (P-TENG)-based triboelectric food-quality sensor (TFQS). (b) Working principles of the TFQS. The self designed graphical user interface (GUI) connected to the TFQS that indicates the crackers are in (c) good or (d) bad condition to eat. PET=polyethylene terephthalate; ITO=indium tin oxide; PTFE=polytetrafluoroethylene; PG-50=pectin-glycerol film whose glycerol content weighs 50% as much as its pectin content.

FIG. 8. COMSOL simulations of the electrical potential of a pectin-glycerol film-based triboelectric nanogenerator with the distance between the two triboelectric layers being I. 1 mm and II. 10 mm.

FIG. 9. Outputs of pectin-glycerol film-based triboelectric nanogenerators made from films with five different proportions of glycerol.

FIG. 10. Output stability of triboelectric nanogenerators made from pectin-glycerol films whose glycerol content weighs 70% as much as their pectin content (i.e., PG-70 films).

FIG. 11. Voltage curves of triboelectric nanogenerators made from pectin-glycerol films whose glycerol content weighs 70% as much as their pectin content (i.e., PG-70 films).

FIG. 12. Variation in the dry-basis moisture content of PG-50 film (i.e., pectin-glycerol film whose glycerol content weighed 50% of its pectin content) when stored at 16% relative humidity over 72 h.

FIG. 13. Digital photographs showing changes in the appearance of a piece of semi-transparent PG-50 film (i.e., pectin-glycerol film whose glycerol content weighed 50% of its pectin content) that was attached to a piece of copper film and stored at 16% relative humidity (RH) for 72 h, then at 99% RH for 72 h.

FIG. 14. Voltage outputs of triboelectric nanogenerators made from PG-70 film (i.e., pectin-glycerol film whose glycerol content weighed 70% of its pectin content) stored at five different relative-humidity levels. Asterisks indicate that, at a given relative-humidity level, there was a significant difference between the voltage outputs after the water-sorption and water-desorption processes (p<0.05).

### DETAILED DESCRIPTION

The subject disclosure describes, among other things, illustrative embodiments for a triboelectric device that can operate as a sensor (e.g., moisture content and/or food quality sensing) and/or an energy harvester.

In one embodiment, the triboelectric device (often referred to as a TENG) can be made from a pectin carbohydrate film with glycerol and water. The concentrations can alter the reflective properties, such as 70% glycol/pectin creating a stronger signal for energy harvesting and 50% glycerol/pectin creating a moisture sensor (due to more distinct variations in the generated signal due to moisture content).

One or more embodiments include a food monitoring device comprising a triboelectric sensor including a film comprising a carbohydrate polymer and a plasticizer. The carbohydrate polymer includes pectin and the plasticizer includes glycerol, where the concentration of glycerol is approximately 50% by weight relative to the pectin, and where the triboelectric sensor is configured to monitor food according to an electrical signal that is generated and which varies based on moisture content, thereby providing real-time monitoring of food quality.

One or more embodiments include a triboelectric device comprising a device body including a carbohydrate polymer, a plasticizer, and multiple layers of material. The carbohydrate polymer includes pectin and the plasticizer includes glycerol, where the concentration of glycerol is approximately 50% by weight relative to the pectin if the triboelectric device is being used as a sensor or is approximately 70% by weight relative to the pectin if the triboelectric device is being used as an energy harvester, and where when mechanical energy is applied to the device body electrical energy is generated.

One or more embodiments include a method comprising determining a concentration for a carbohydrate polymer and a plasticizer to be used in a triboelectric device, where the triboelectric device is to be used as one of a sensor or an energy harvester, where the concentration is different depending on usage as the sensor or the energy harvester, and where the determining the concentration is based on improving operation as the sensor or improving operations as the energy harvester. The method can include providing the triboelectric device including the polymer and the plasticizer with an object that is subjected to mechanical energy, where the triboelectric device comprises multiple layers of material including a conductor and an insulator. The method can include connecting the triboelectric device to an electrical component, where when the mechanical energy is applied to the device body electrical energy is generated and provided to the electrical component.

Triboelectric nanogenerators (TENGs) are a class of devices that can convert ambient mechanical energy into electricity based on the combination of contact electrification and electrostatic induction. Because contact electrification can occur when almost any two different materials come into contact, the range of potential materials that can be used in TENGs' construction is virtually limitless, meaning that they can be endowed with various useful properties such as flexibility, biodegradability, transparency, and so forth. Among TENGs' many other advantages include versatility—their electrical output can be used directly as a power source or serve as signals for sensing—as well as low cost and ease of fabrication.

More importantly, as compared to their main rivals, electromagnetic generators, TENGs are more effective at harvesting low-frequency (<4 Hz) mechanical energy, which is more commonly encountered in nature (e.g., human motion) than its higher-frequency counterpart.

In general, TENGs' output performance decreases as RH increases. This could be due to water-related interactions on their triboelectric layers under high-RH conditions, which may affect their accumulated triboelectric charges undesirably. However, different materials interact with water molecules differently. For example, it was found that a TENG using film made from starch—a polysaccharide—had its voltage output rise as RH increased from 15% to 95%. This was possibly due to change in the triboelectric properties and/or surface structure of the starch film as it adsorbed or absorbed moisture when placed in higher RH conditions. Similarly, a gelatin—a protein—used when fabricating TENGs, resulted in devices' voltage output increasing as RH increased from 20% to 60%. However, the voltage output decreased when RH increased from 60% to 80%.

In one or more embodiments described herein, a carbohydrate polymer, e.g., pectin, that is commonly extracted from apple and citrus fruit-peel waste can be utilized as a core material for TENG fabrication. Because, as noted above, TENGs' electrical output can be used both as power and as sensing signals, pectin-based TENGs' can have a capability for both energy harvesting and RH monitoring. Other carbohydrates or other materials can be used. For example, in place of or in combination with pectin other materials that can be used in TENGs include other carbohydrate polymers and natural polymers that may offer similar properties. Some potential materials are: Starch: A widely available and biodegradable polymer that can be used in TENGs. It has triboelectric properties and can be modified to enhance performance. Cellulose: An abundant natural polymer known for its mechanical strength and flexibility, suitable for use in TENGs. Chitosan: Derived from chitin, this biodegradable polymer can be used in various applications, including TENGs. Agar: A gelatinous substance derived from seaweed, agar can be used as a biopolymer in TENGs due to its gel-forming properties and biodegradability. Guar Gum: A polysaccharide extracted from guar beans, guar gum can be used to create flexible films suitable for TENG applications. Xanthan Gum: A polysaccharide used as a food thickening agent, xanthan gum can be utilized for its film-forming capabilities in TENGs. Other potential materials or options may include carrageenan, alginate, dextran, pullulan, xylan, and glucan, among others. These materials can be selected based on the desired properties of the TENG, such as flexibility, biodegradability, and triboelectric performance. The choice of materials will depend on the specific application and the performance requirements of the TENG, which may include the type of food item that is being monitored.

Plasticizers are commonly used in the production of biopolymer-based films to endow them with flexibility and/or increased moldability. Therefore, different concentrations of plasticizer, e.g., glycerol, can be used to affect the triboelectric performance of films such as pectin films. Other types of plasticizers can also be used. In combination with or in place of glycerol, other materials that can be used as plasticizers in TENGs include other substances that enhance the flexibility and mechanical properties of biopolymer films. Some potential materials are: Sorbitol: A sugar alcohol that can be used as a plasticizer to improve the flexibility and processability of carbohydrate films. Polyethylene Glycol (PEG): A common plasticizer that can enhance the flexibility and mechanical properties of biopolymer films. Propylene Glycol: Another plasticizer that can be used to modify the mechanical properties of carbohydrate-based films. Vegetable Glycerin: Similar to glycerol, vegetable glycerin is derived from plant oils and can be used as a plasticizer to enhance flexibility. Lecithin: A natural emulsifier found in egg yolks and soybeans, lecithin can be used to improve the flexibility and processability of biopolymer films. Maltodextrin: A polysaccharide used as a food additive, maltodextrin can act as a plasticizer to modify the mechanical properties of carbohydrate-based films. These materials can be selected based on their availability, cost, and the specific triboelectric properties desired for the TENG application. The choice will depend on the intended use and performance requirements of the TENG, which may include the type of packaging material that the TENG is to be utilized with, such as cardboard vs a plastic bag.

Optimization or improvement of concentration of glycerol can be performed in TENGs, which is a key component for synthesizing pectin films. The effects of pectin films' glycerol concentrations on triboelectric performance can be leveraged as described herein. More importantly, as pectin molecules and a plasticizer, e.g., glycerol, both possess a hygroscopic property, the unique interaction between pectin/glycerol films and water molecules can be leveraged to develop TENGs for humidity monitoring, which would be of considerable value in food-quality monitoring, including for the reasons discussed above. As such, one or more of the embodiments described herein can leverage a variation in the glycerol content of triboelectric devices to make them better suited to specific applications.

In one embodiment, a film with a determined glycerol content that had led to the highest TENG output was used to assess the energy-harvesting capabilities of these devices. This included evaluating their peak power output and effectiveness at harvesting mechanical energy to power portable electronics. It was further determined whether pectin films with different glycerol content, when stored at various RH levels, led to different levels of triboelectric-sensor performance. This information allows developing a range of triboelectric RH sensors able to provide information linked to various food products.

In one embodiment, a low-moisture food, e.g., crackers, can be monitored which is based on different RH storage conditions affecting quality. For instance, it can be shown that key quality attributes (e.g., moisture content and hardness) will change when such storage conditions changed, both from low to high (i.e., water sorption) and from high to low (i.e., water desorption).

In one embodiment, output data from triboelectric sensors made from pectin films can be matched with varied glycerol content to the resulting cracker quality-attributes data (or other food being evaluated for food quality data as a function of moisture content). A user-friendly graphical user interface (GUI) can be provided that utilizes a pectin-based triboelectric sensor system to indicate the quality of low-moisture food. As TENG-based sensors do not require batteries or other external sources of electricity to generate signals, they can be integrated into intelligent packaging that provides real-time food-quality information directly to consumers. Here, it is worth noting that conventional date labels such as “Best if used by . . . ” are typically based on estimation, not exact science, and do not take storage conditions such as temperature and humidity into account. Therefore, providing evidence based dynamic information about food quality could empower consumers to make informed decisions about when to keep, consume, or discard food products, thus potentially alleviating the heavy economic, environmental, and societal burdens that food waste causes.

Various materials can be utilized for the TENG device such as a Pectin film TENG (i.e., P-TENG). As an example, the materials can include low-methoxyl food-grade pectin powders sourced from citrus peels, glycerol, Milli-Q water with a resistivity of 18.2 MΩ·cm, magnesium chloride hexahydrate (MgCl₂·6H₂O, 98%), magnesium nitrate (Mg(NO₃)2, 98%) and sodium chloride (NaCl, 99%), polytetrafluoroethylene (PTFE) film, copper tape with conductive adhesive, and acrylic boards.

Pectin films with varying glycerol concentrations can be made using various techniques including a solution-casting method. Pectin/glycerol (PG) solutions can be created in which the concentration of pectin is kept at 3 wt %. The glycerol concentration can be determined based on its weight relative to the weight of pectin added to the solution. For example, “PG-10” is the designation for films in which the added glycerol had weighed 10% as much as the pectin in the solution. The other five formulations, named according to the same convention, were PG-0, PG-30, PG-50, PG-70 and PG-90.

The four glycerol-containing solutions (i.e., PG-30, PG-50, PG-70 and PG-90) and the one without glycerol (i.e., PG-0) can be heated, such as at 50° C., with stirring, such as at 800 rpm for 15 min, followed by the addition of pectin powders into each solution. Each can be mixed, such as at 800 rpm at 50° C., until the pectin is fully dissolved. Next, they can be allowed to cool at room temperature for 1 hour (h), with lids on to prevent moisture evaporation. The cooled PG solutions can be degassed with a vacuum pump. Then, 40 mL of each degassed PG solution can be poured onto glass Petri dishes, which can be put in a convective oven for drying, such as at 50° C. for 24 hours. After drying, the pectin films can be peeled off the glass Petri dishes.

In this example, the surface topographies of PG-0 and PG-50 film were characterized by atomic-force microscopy (AFM) in tapping mode. The moisture content of each type of PG film was measured with a moisture-content analyzer. This can be done by, first, trimming of all PG-50 films into 3 cm×3 cm squares, which were then stored at 16% RH for 3 days to ensure consistent initial moisture content. Next, to observe changes in the moisture content of films at different RH-level storage conditions over time, the samples were divided into five equally sized groups. One of these groups was kept at 16% RH, while the remaining four were transferred to 33% RH, 55% RH, 75% RH and 99% RH, respectively. Their moisture content was measured in triplicate immediately before they were placed in storage and again after 1 h, 4 h, 8 h, 24 h, 32 h, 52 h, and 72 h. During those measurements, the above-mentioned moisture-content analyzer was used. Afterwards, the dry-basis moisture content was used for data analysis.

Fourier-transform infrared (FTIR) spectra of PG-50 films stored at the five different abovementioned RH levels were characterized using an FTIR spectrometer equipped with attenuated total reflection (ATR). After storage, PG-50 films were transferred for FTIR analysis from 4000 to 400 cm-1 at a resolution of 4 cm-1.

In one embodiment, the P-TENGs can mainly consist of two triboelectric layers—one made from PG film and the other from PTFE (or other suitable material)—each with a copper layer (or other suitable conductor) attached to it to serve as a conductor, on an acrylic substrate. In this example, to evaluate the P-TENGs' output and assess the triboelectric performance of their corresponding PG films, one of these triboelectric layers was placed on a programmable linear motor and the other was mounted on an L-shaped stand. When the linear motor was operated, the two layers came into contact and separated periodically. The P-TENGs' electrical outputs were characterized using an electrometer and a LabView based data-display system. COMSOL Multiphysics software was used to simulate the changes in electrical potential between the two triboelectric layers that would occur when the distance between them changed.

In this example, to assess the impact of storage-RH levels on quality change in crackers, the cracker samples can be divided into two parts: a moisture-sorption group and a moisture-desorption group. In the former, crackers were initially kept at 16% RH for 3 days (d), and then for a further 3 d either maintained at 16% RH or transferred to one of the following four RH levels: 33%, 55%, 75% or 99%. In the moisture-desorption group, on the other hand, each cracker was first kept at 99% RH for 3 d, then transferred and stored for 1 d to one of the same five RH-level conditions listed above. Crackers' quality was assessed via measurement of their moisture content and hardness.

Specifically in this example, moisture content was measured using the same moisture-content analyzer mentioned above. Measurements of hardness were adapted from formerly published methods. Specifically, a TA-18 probe (Texture Technologies, Hamilton, MA, USA) operated by a Stable Micro System texture analyzer (TA) (Texture Technologies, Hamilton, MA, USA) was used for such measurements. Briefly, a piece of cracker to be measured was placed in the center of a stage before force was applied. The initial distance between the probe and the cracker was set at 20 mm. Probe speed during measurement was set at 1 mm·s-1, and deformation at 3 mm. The triboelectric food-quality sensors (TFQSs) mainly consisted of 1) a 5 cm×5 cm PG-50 film attached to a piece of indium-tin-oxide (ITO)-coated polyethylene terephthalate (PET) film and 2) the same size film, but made of PTFE, attached to ITO-coated PET film. PG-50 and PTFE served as the two triboelectric layers; the ITO, as the electrode layer; and the PET, as the substrate.

The two main parts were then attached to the inner surfaces of the top and bottom walls, respectively, of a cracker-packaging box, with the PTFE and PG film facing each other. To demonstrate the resulting TFQS's ability to monitor the quality of food, the TFQS was first connected to the electrometer mentioned above, which in turn was connected to a computer with a GUI. A model was built or otherwise generated by matching change in the hardness of the crackers to the output performance of the optimal TFQS, and the GUI (according to the model data) was able to display the quality of the crackers whenever the two triboelectric layers in the cracker box were manually pressed and released. The box can be made of various materials including paper or plastic.

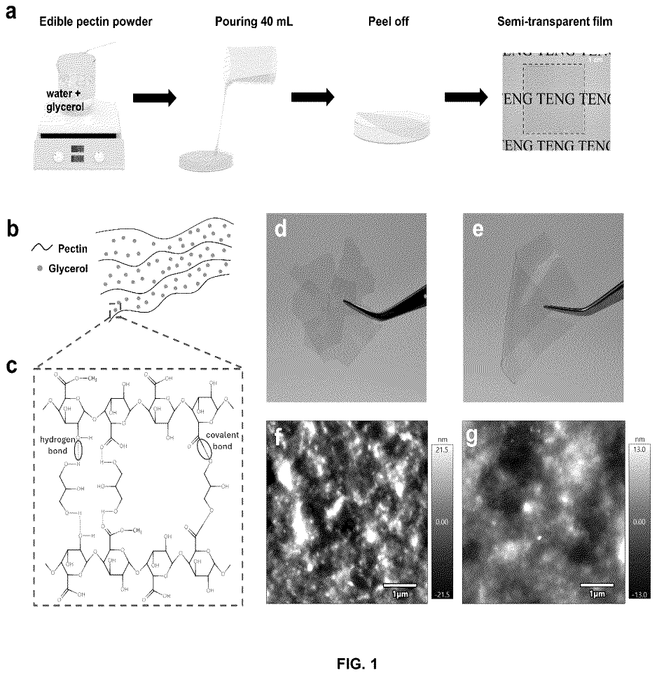

FIG. 1a shows the synthesis process of PG films, which were semi-transparent with a light yellowish color. As shown in FIGS. 1b and 1c, the molecules of the glycerol added to these films as a plasticizer could form hydrogen bonds or covalent bonds with pectin chains. Specifically, hydrogen bonds could be formed between the hydroxyl groups on glycerol molecules and the protonated carboxyl groups or hydroxyl groups on adjacent pectin chains; whereas covalent bonds could be formed via esterification reactions between the glycerol hydroxyl groups and carboxyl groups on neighboring pectin chains. The formation of these bonds could solvate the hydrophilic sites on pectin chains, reducing intermolecular attraction between neighboring pectin chains. As a result in this example, films containing glycerol (e.g., PG-50) were less brittle, more flexible and showed better mechanical properties than their no-glycerol counterpart, PG-0. For instance, FIGS. 1d and 1e show that PG-50 film exhibited excellent flexibility, while PG-0 film displayed a brittle property, fracturing when bent.

The addition of glycerol also altered PG films' surface topography. FIGS. 1f and 1g are AFM images scanned across the surfaces of PG-0 and PG-50 films, respectively. When no glycerol had been added to a film, its surface included many protuberant “beads”, which can be observed as white regions in FIG. 1f These structures are likely to have resulted from the aggregation of hydrophilic regions among pectin molecules. Comparatively, the surface of PG-50 film was smoother (FIG. 1g), displaying fewer beads and more uniform height distributions. This was probably because the plasticization effects of the glycerol had alleviated intermolecular attractions among the pectin's molecular chains. Similar phenomena were previously observed when glycerol was added to other polysaccharide-based films.

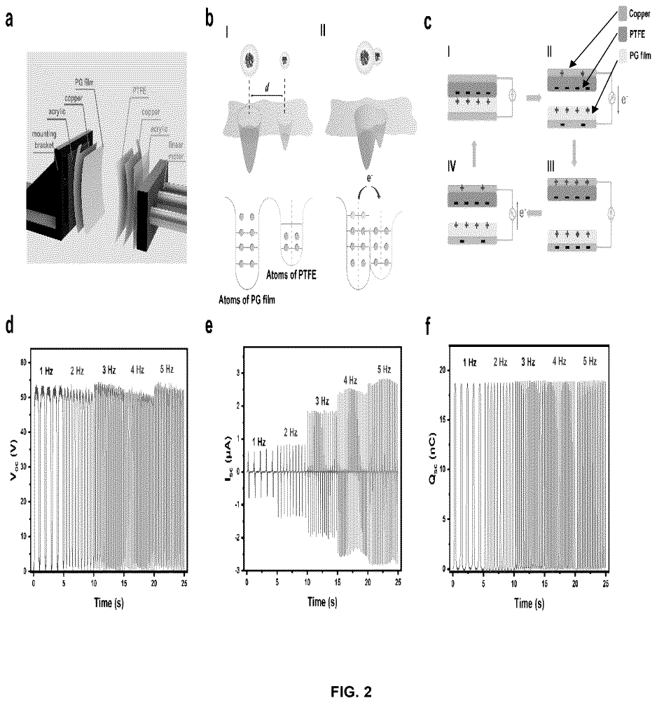

All P-TENGs were operated in contact-separation mode, and their structure is shown in FIG. 2a. Prior to contact between their PG film and PTFE triboelectric layers, the electron clouds of both materials, formed by their outer-shell electrons, remain separated by a distance of d (FIG. 2b-I). When the two triboelectric layers come into contact, their electron clouds overlap, enabling electron migration between them and resulting in electrification (FIG. 2b-II).

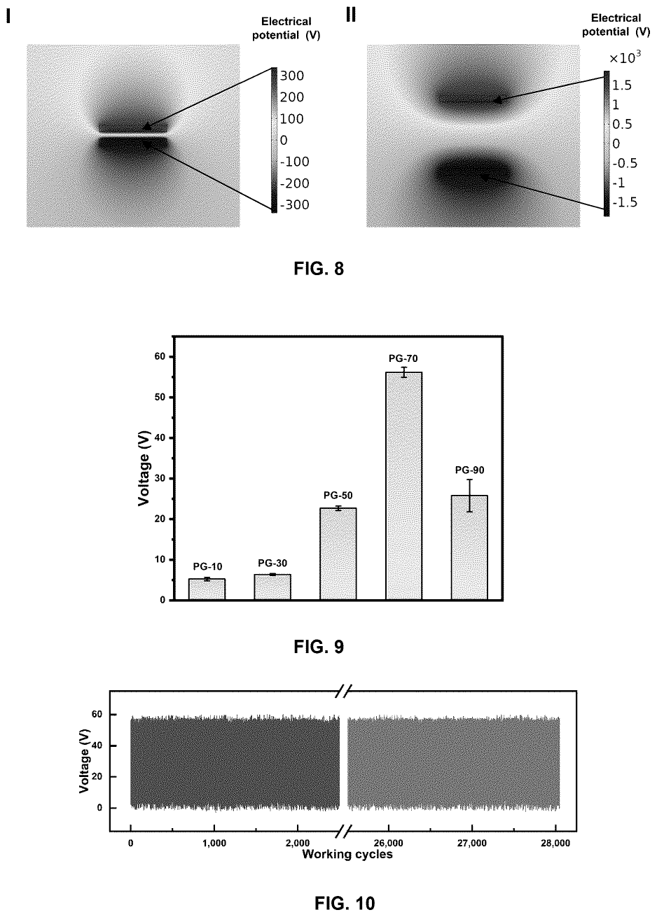

Because of the greater electronegativity of PTFE, negative charges occur on PTFE and positive ones occur on PG film. Similarly, as shown in FIG. 2c-I, when the PG film and PTFE come into contact due to the application of external force, the overlapping of their respective electron clouds results in equal but opposite charges on their respective surfaces. Then, when the external force ceases and they begin to separate, due to electrostatic induction, opposite charges are induced on the electrode layers attached to the two triboelectric layers, causing electrons to flow from the electrode attached to the PTFE layer to the electrode attached to the PG film layer (FIG. 2c. III) until they are fully separated (FIG. 2c. IV). The COMSOL simulation results shown in FIG. 8 also indicate that electrical-potential difference increases as the distance between them increases. Then, when they come closer again, an electron flow in the opposite direction can be observed (FIG. 2c-V), and lasts until the two triboelectric layers are in contact with each other again.

As glycerol content can greatly affect the properties of biopolymer-based films, varying glycerol content can be monitored for impact on the triboelectric performance of the resulting devices. As can be seen from FIG. 9, at higher glycerol content levels ranging from PG-10 through PG-70, P-TENGs' electrical output increased. This was likely because increasing the glycerol content led to the presence of more hydroxyl groups (from glycerol molecules) that could serve as electron donors, thus leading to higher output when the resulting films came into contact with PTFE. However, at a higher concentration of glycerol (i.e., PG-90), the electrical output of the devices was lower. This could have been because such higher concentration facilitated water sorption, and the sorbed water might have neutralized triboelectric charges, resulting in lower triboelectric output. As a result, the PG-70 films might be considered (in this example) as the optimal ones for fabricating P-TENG-based energy-harvesters.

The output performance, specifically open-circuit voltage (Voc), shirt-circuit current (Isc) and short-circuit charge (Qsc), of a P-TENG incorporating PG-70 film are shown in FIGS. 2d-2f. It was observed that when frequency increased from 1 Hz to 5 Hz, peak values of Voc and Qsc remained at almost the same level, with the former being approximately 55 V and the latter being about 19 nC. This was because Voc was related to two factors: 1) the charge density induced on the contact surfaces, as well as the distance between PG70 film and PTFE during contact and separation, and 2) the total charge transferred in one cycle.

Both these factors remained constant as the frequency changed. However, Isc increased from about 0.8 μA to around 2.9 μA when frequency increased from 1 Hz to 5 Hz. Current (I) can be calculated using Eq. (1), below,

$$I = \frac{dQ}{dt} \qquad ( 1 )$$

where dQ represents the amount of charge transferred during one cycle, and dt denotes the time required for charge transfer in one cycle. Therefore, as the frequency increases, dt decreases, causing a higher I.

In one embodiment, long-term stability is illustrated as shown in FIG. 10 where after approximately 28,000 cycles of continuous operation, the voltage output of one such device remained at a similar level to its original output.

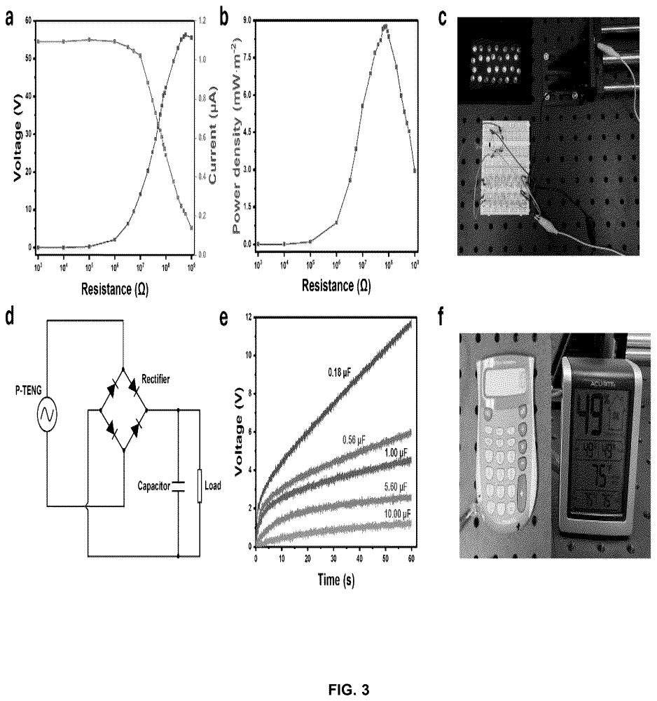

External resistors can be used to evaluate the energy-harvesting performance of a PTENG, such as made from PG-70. As shown in FIG. 3a, as resistance from the external circuit increased from 1 kΩ to 500 MΩ, the device's voltage output also increased. However, when resistance increased further, from 500 MΩ to 1 GΩ, such output remained at around the same level (i.e., around 55 V). The probable reason was that, in the latter case, the resistance had become so large that it created a condition similar to an open-circuit one, leading to output performance that was close to the Voc values we measured. On the other hand, as resistance increased from 1 kΩ to 1 GΩ, current output decreased from around 1.0 μA to about 0.1 μA.

The power density of a P-TENG can be calculated using Eq. (2),

$${P}_{d} = ( {I}^{2} \cdot R ) / A \qquad ( 2 )$$

where Pd is power density, I is the measured current, R is the resistance, and A is the area of the contact surface. The relationship between power density and resistance is shown in FIG. 3b, and maximum power density, 8.75 mW·m-2, was reached when R was 80 MΩ. This demonstrates that the creation of effective pectin-based TENGs for energy harvesting can be achieved by chemical syntheses of PG films and assembly processes.

Moreover, by adjusting their concentrations of glycerol, the performance of such TENGs can be optimized. This should be useful to anyone aiming to design high-performance TENGs based on pectin or another carbohydrate polymer.

P-TENG-based energy-harvesting devices can be created for various practical applications. For example, a P-TENG made from PG-70 film was connected to a full-bridge rectifier that could convert its AC output into direct current (DC). As can be seen from FIG. 3c, simple hand tapping could light up to 27 light-emitting diodes (LEDs) connected in series. This result not only suggests that P-TENGs can convert mechanical motion into electricity, but also that such devices could be used in environments with repeated mechanical motions, such as interactive gaming.

The same PG-70 film-based TENG also has the ability to power portable electronics. Specifically in one example, it was connected in a circuit first to a full-bridge rectifier and then to a 50 μF capacitor (FIG. 3d). The DC output converted by the rectifier charged the capacitor, which could then be used as a power source. FIG. 3e, which shows the charging rates of the P-TENG using different capacitors when operated at a frequency of 5 Hz, indicates that the smaller the capacitance, the faster the charging rate. For example, the 10 μF capacitor was charged to 1.2 V within 60 sec of TENG operation, whereas the 0.18 μF capacitor reached 11.6 V in the same timeframe.

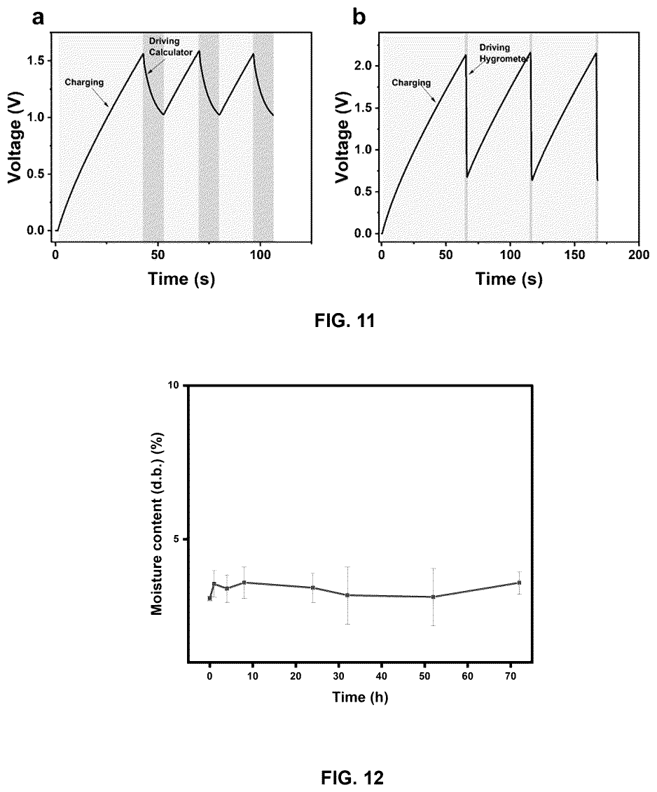

The same above-mentioned P-TENGs with power-management circuits were then evaluated in terms of their capability to power small electronics. FIG. 3f and FIG. 11 show that a calculator and a digital hygrometer were successfully powered up by this system when mechanical energy was applied to it. These results indicate a P-TENG made of PG-70 film can successfully convert mechanical energy into sufficient electrical power for many small electronic devices in common use.

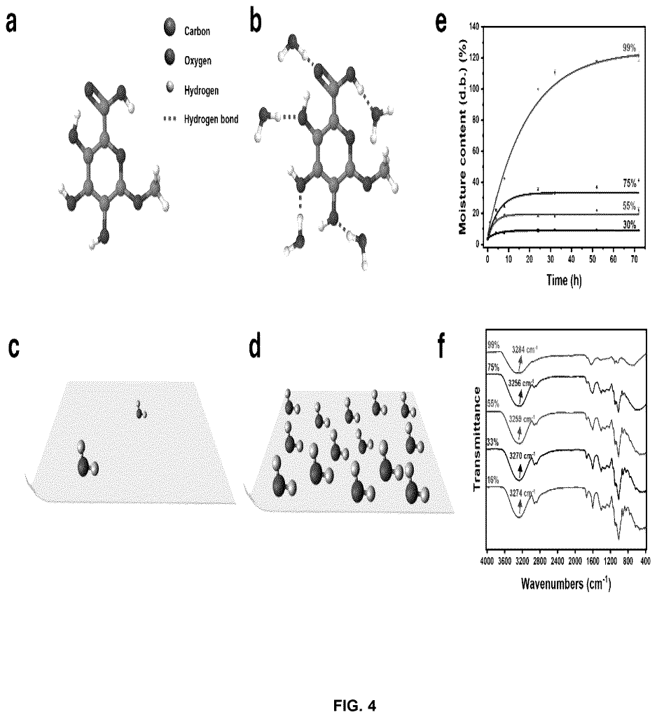

The backbones of pectin are mainly composed of D-galacturonic acid (GA) molecules, which link via α-(1,4)-glycosidic bonds by hydroxyl groups on C1 and C4. The molecular structure of GA is shown in FIG. 4a. Functional groups on GA molecules, such as hydroxyl and carboxyl groups, can potentially form hydrogen bonds with water molecules, as illustrated in FIG. 4b. Additionally, as noted above, glycerol molecules possess a hygroscopic property.

Therefore, water molecules in the environment can be sorbed by PG-50 films. FIGS. 4c and 4d respectively demonstrate the water-sorption behavior of PG-50 films at lower and higher RH levels. PG-50 films' water sorption behavior can be determined at different RH levels. Similarly, pectin/glycerol-water interactions can inform a design of triboelectric sensors. FIGS. 4e and 12 show how PG-50 films' moisture content changed when they were stored at different RH levels over the course of 72 h (i.e., at 33%, 55%, 75, and 99% RH for the former figure and 16% RH for the latter). As noted above, prior to such storage, the PG-50 films were first kept at the lowest RH level of the five (i.e., 16% RH) for 3 d. FIG. 12 demonstrates that PG-50 films in the 16% RH storage condition had barely sorbed water—as indicated by the negligible change in their moisture content—over the course of 72 h storage, likely due to the low quantity of water molecules in that environment. However, FIG. 4e shows that when RH levels were 33%, 55% and 75%, the moisture content of the PG-50 films increased, up to a point, as storage time increased. But after approximately 24 h of storage at those RH levels, such content stabilized at 8.9%, 18.1% and 35.2% respectively for the three RH levels.

In general, at the same temperature, higher RH levels suggest that the environment contains more water molecules. Consequently, at those levels, more such molecules could potentially be sorbed by the PG-50 films, which might explain the above findings regarding PG-50 films' moisture content increasing alongside RH levels. Conversely, the reason pectin films' moisture content remained unchanged from the 24-h to the 72-h mark at all three of the tested RH levels from 33% to 75% was that the amount of water molecules in the storage containers was still limited, and thus, such films' water sorption was able to reach equilibrium.

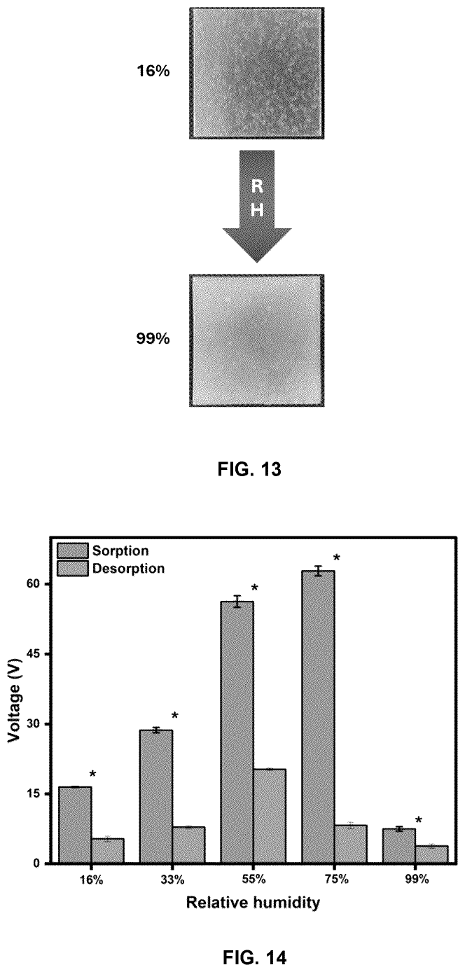

However, in ultra-high (99%) RH storage conditions, water molecules in the environment were so abundant that the moisture content of PG-50 films increased drastically, from 6.0% to 121.7% after 72 h of storage (FIG. 4e), and never reached equilibrium during that period. This implies that, above a certain RH level, sorbed water molecules could occupy all the sites on pectin films where water molecules might form hydrogen bonds. In that state, any excess water molecules in the environment could still be physically sorbed by the film, but they would likely appear in the microcapillaries and voids on its surfaces. Accumulation of excess water could also be observed visually. As shown in FIG. 13, as compared to a P-TENG stored at 16% RH whose PG-50 film was clear, flat, and dry, PG-50 film stored at 99% RH was opaque and swollen.

FIG. 4e also includes the fitting curves of the experimental data derived from the model:

$$u = a + b ( 1 - {exp}^{( - c τ )} ) \qquad ( 2 )$$

where a, b and c are constants of the equation; z is storage time (h); and u is the dry-based moisture content (%) of the pectin films (i.e., PG-50 in this case). The u can be expressed as

$$u = \frac{{M}_{w}}{{M}_{d}} \times 100 \% \qquad ( 3 )$$

where Mw is the weight of water in a PG-50 film sample and Md is the weight of the dry matter in that sample. The curve-fitting results show that our experimental data fit that model well (R2>0.96).

Importantly, as the moisture content of the films changed over time, either before reaching equilibrium (in the case of 33%, 55% and 75% RH) or continuously (in the case of 99% RH), they might contain “histories” of moisture changes rather than providing real-time information corresponding to their storage conditions' RH levels. For example, a typical real-time electronic RH sensor might give the same result for hour 2 and hour 10 of storage, whereas a sensor based on PG-50 film could reveal different, and more accurate, results for those same time points. Such “history” information could be of great importance in future approaches to food-quality monitoring, especially given that much food-quality degradation is irreversible.

In this example, FTIR spectroscopy was further used to probe potential chemical interactions between PG-50 films and water molecules. FIG. 4c shows the FTIR spectra of such films stored under different RH conditions for 72 h. In the spectrum of the PG-50 film stored at 16% RH, the broad absorption peak near 3,274 cm-1 could be attributed to the O—H stretching vibration peak. The peak located at 2,924 cm-1 could be ascribed to the C—H stretching vibration of methylene groups; the one at 1,742 cm-1 may be associated with C═O group in ester stretching; the one at 1,600 cm-1 might correspond to carboxyl groups' asymmetric stretching; and the one at 1,407 cm-1 is likely due to such groups' symmetric stretching. Lastly, the peak at 1,008 cm-1 is likely associated with C—O and C—C stretching vibrations.

It is critical to point out that when storage RH levels were higher than 16%, the O—H stretching vibration peak of the corresponding PG-50 film differed: i.e., fell from 3,274 cm-1 to lower wavenumbers associated with the O—H stretching vibration of water molecules' hydrogen bonds. Specifically, it was 3,270 cm-1 at 33% RH, 3,259 cm-1 at 55% RH, and 3,256 cm-1 at 75% RH. This was probably because of the formation of hydrogen bonds between water and pectin molecules. However, when RH increased to 99%, the peak changed to a drastically higher wavenumber (3,256 cm-1 at 75% RH→3,284 cm-1 at 99% RH), probably due to physically sorbed water in/on the PG-50 film.

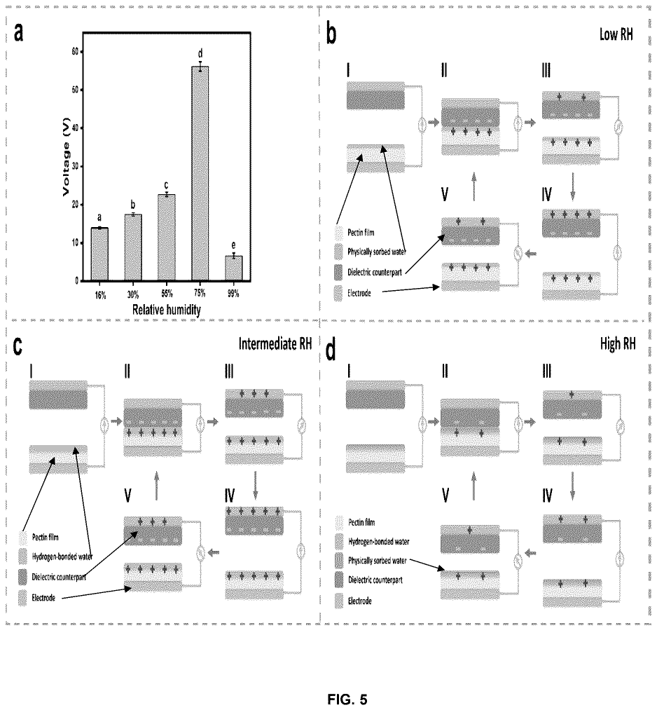

After studying the potential interactions between water molecules and PG-50 films, it was determined how they could influence the output performance of TENGs incorporating such films. As shown in FIG. 5a, after storage at five different RH levels for 3 d, PG-50 film-based PTENGs had markedly different voltage outputs. Specifically, after storage at 16% RH for 3 d, output was around 14 V. At storage RH levels of up to 75%, on the other hand, it was significantly higher (p<0.05): i.e., 17 V at 33%, 23 V at 55%, and 56 V at 75%. However, when RH increased to 99%, output dropped significantly (p<0.05), to 6 V. These phenomena were likely due to the above-mentioned variation in interaction between water molecules and pectin films, which could affect contact-electrification between the latter and their triboelectric counterparts.

FIGS. 5a-d depict various mechanisms that could have caused the output variations observed in P-TENGs that had been stored at various RH levels. First, the thin green layer in FIG. 5b is used to represent water molecules forming hydrogen bonds with pectin molecules when RH is low (i.e., 16%). As the overall number of water molecules in a 16%-RH storage environment could be quite limited, the amount of hydrogen-bonded water on/in the pectin films is therefore sporadic, and leads to lower electrical output.

After storage in intermediate-RH environments (i.e., between 33% and 75%), where the numbers of available water molecules are greater, more water could be sorbed by PG-50 film, and water molecules would be able to form more hydrogen bonds with pectin molecules. This is depicted in FIG. 5c as a thicker green layer. In this circumstance, due to hydrogen bonding between water molecules and pectin molecules, the former could become part of the pectin film's structure. Water, like pectin, is a material inclined to lose electrons during contact-electrification. Thus, a pectin layer with a high moisture content could be more triboelectrically positive than its low-moisture counterpart (FIG. 5c), and this could lead to higher output when it comes into contact with a triboelectrically negative layer such as PTFE.

When RH was even higher (e.g., 99%), the formation of hydrogen-bonding water on PG-50 films could became saturated; i.e., all the active sites for hydrogen bonding on the pectin films might be occupied. Again, the excess water molecules could be further physically sorbed into/onto the films. Unlike hydrogen-bonding water, accumulation of physically sorbed water could lead to the formation of a water layer on PG-50 film that could neutralize the charges during contact-electrification and thus greatly reduce the output of any TENG made from such film. This physically sorbed layer is depicted in blue in FIG. 5d.

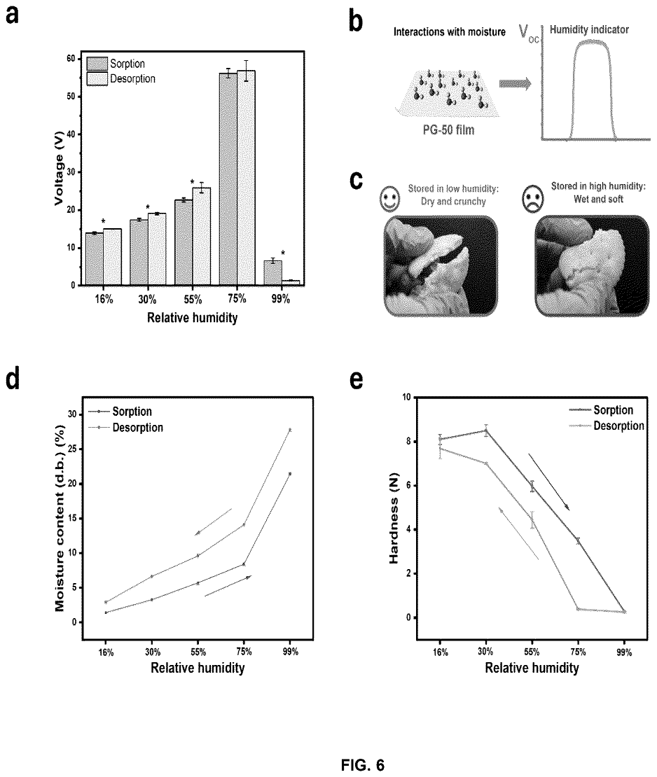

It is important to point out that, in the embodiment described above, PG films were first stored at a low RH, i.e., 16%, before being exposed (in four out of five cases) to higher RH levels. As the moisture content of the PG films after their 16% RH exposure was low, the process that ensued from placing them into higher-RH environments is denoted herein as “sorption”. “Desorption” process is denoted for PG films that were first stored at a high RH level (i.e., 99%) for 3 d and then either kept at that RH level or moved to lower ones for an additional 1 d. Interestingly, FIG. 6a shows that, even in the desorption process, the voltage outputs of our P-TENGs were significantly higher (p<0.05) at higher final RH storage levels: i.e., approximately 57 V at 75% RH vs. 26 V at 55% RH vs. 19 V at 33% RH vs. 15 V at 16% RH.

However, in line with results determined earlier, the P-TENG that was kept at 99% RH throughout the desorption process had the lowest output of the five (1.4 V). This was likely because more water physically formed on/in its PG-50 film, leading to reduced triboelectric output, similarly to the phenomenon observed in the sorption process.

More importantly, however, the differences in PG-50-based P-TENGs' voltage output differed significantly (p<0.05) across the data obtained from the sorption and desorption processes, at all levels except 75% RH. This is potentially important for anyone creating food quality monitoring sensors, as food products in general exhibit hysteresis between moisture sorption and desorption.

Despite this statistically significant difference between our sorption and desorption data, the voltage outputs of the P-TENGs made of PG-50 film could be grouped into ranges that may be practically useful: that is, for 99%, between 1 V and 9 V; for 16% RH, between 10 V and 16 V; for 33% RH, between 17 V and 20 V; for 55% RH, between 21 V and 25 V; and for 75% RH, between 50 V and 60 V. Such ranges could potentially represent the RH levels at which food products are stored and matched to food-quality changes, which typically occur gradually. FIG. 6b shows P-TENG's potential as a humidity sensor. When stored in a certain RH level, PG-50 film interacts with water molecules in such condition, endowing P-TENG an output that lies in a specific range which indicates the storage humidity level.

But interestingly, when PG-50 film was replaced with PG-70 film (P-TENGs made from which had the highest voltage output of any tested in this example), this characteristic no longer held, as shown in FIG. 14. That is, the voltage outputs of P-TENGs incorporating PG-70 film could not be grouped into representative voltage ranges, except in the case of those with films that had been stored at 99% RH. This discrepancy was probably because the increased amount of glycerol in PG-70 films led to deviation in their hygroscopic properties, as compared to those of PG-50 films; and this, in turn, could have caused different sorption and desorption behaviors when they were interacting with water molecules. In a broader sense, these results also highlight that changing the glycerol concentrations of biopolymer films could endow triboelectric devices made from them with greater versatility.

Based solely on the observation that voltage output during the sorption process increased significantly (p<0.05) as storage RH increased from 16% to 75%, one could still argue that the devices fabricated using PG-70 films could function as humidity sensors. However, some data from the desorption process may contradict that premise: i.e., their voltage output only increased significantly (p<0.05) when the final desorbed RH was between 16% and 55%. And perhaps more importantly, the sorption and desorption data were not similar enough to confidently associate any particular voltage ranges with any practically relevant humidity information.

Therefore, the PG-70 may be less suitable or even unsuitable for such humidity detection applications. Also, given that many food products and biopolymers undergo hysteresis between moisture sorption and desorption, both of these processes should be considered when seeking accurate data on moisture, humidity, and/or food quality.

Crackers—a low-moisture food whose quality can be greatly affected by its storage environment—were selected as a target in one example of PG-50 film-based P-TENGs' potential as food quality monitors. Quality changes can be determined in crackers stored at the same five RH levels discussed above, by evaluating two of their key quality attributes: hardness—the peak force applied to snap the crackers—and moisture content. As shown in FIG. 6c, when storage RH was low (i.e., 33% or below), crackers remained dry and crisp; but when stored under high-RH conditions (i.e., 55% or above), they became soggy and soft. The crackers can then be subjected to the same sorption and desorption processes that the P-TENGs were previously exposed to and then characterized the same two quality attributes again.

This established that, after the sorption process, the crackers' moisture content increased significantly (p<0.05) alongside the increase in storage RH levels; i.e., these food items were absorbing more water as RH went up. Conversely, during desorption, the crackers' moisture content decreased significantly (p<0.05), indicating that they were releasing water into the environment as storage RH level fell. Importantly, however, the final moisture content of the crackers after desorption always ended up significantly higher than that after sorption (p<0.05) at any given RH level, implying possible hysteresis between these two processes. This reaffirms the potential of PG-50 P-TENGs for monitoring the quality of low-moisture foods.

Unsurprisingly, given that moisture content can profoundly affect food quality, crackers' other key quality attribute—hardness—exhibited a similar trend. Their hardness after storage at 16% and 33% RH did not significantly differ (p>0.05). However, as shown in FIG. 6e, after undergoing sorption as RH levels rose from 33% to 99%, their hardness dropped significantly (p<0.05). Then, following desorption, they recovered some hardness, but never to the original level: further possible evidence in favor of hysteresis occurring between this food product's sorption and desorption of moisture.

In line with PG-50 P-TENG data, quality-attributes data can also be grouped for practical purposes. Specifically, in the cases of both sorption and desorption, if final RH storage levels were lower (i.e., 16% or 33%), crackers' hardness remained above 6.5 N. If the final RH storage level was 55%, the hardness of the resulting crackers in the case of both sorption and desorption fell within the range of 4.0 to 6.5 N; and such values dropped below 4.0 N when final RH storage levels were 75% or higher.

Based on these quality-attributes data, a GUI can be created that integrates both sets of grouping data, i.e., one set pertaining to PG-50 P-TENGs (or other types of TENG having concentrations that allow for monitoring as described herein) and the other to cracker hardness. Additionally, based on the same preliminary data, a triboelectric food-quality sensor (TFQS) can be created and incorporated into food packaging, such as a cracker box containing crackers, which when stored at various RH levels, allows for reading out the quality of the crackers using the above-mentioned GUI.

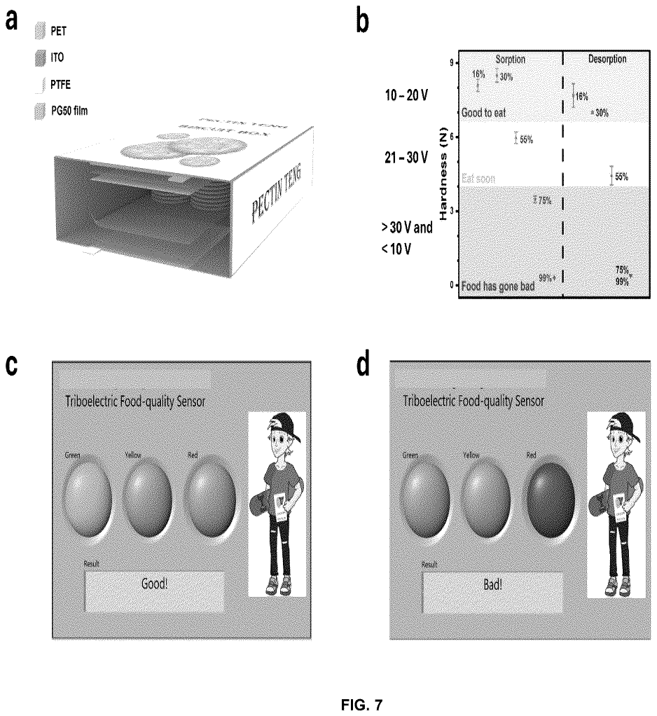

Specifically in this example, as shown in FIG. 7a, the TFQS comprised a P-TENG with PG-50 film and PTFE as its two triboelectric layers, each of which was attached to a layer of ITO-coated PET, with the ITO serving as an electrode and the PET as a flexible substrate. The two resulting composite layers were respectively attached to the inner surfaces of the top and bottom walls of the cracker box, facing each other. Thanks to its P-TENG's thin structure, the TFQS was lightweight, highly portable, and readily integrable into this style of food packaging. By simply pressing and releasing the box with a user's hands, the TFQS can generate voltage outputs, which the GUI then processes and links to food quality. It should be noted that, in practice, substrates can be added to each of the two triboelectric layers before placing them in such packaging, to reduce the distance between the layers and thus minimize the need for deformation of the box when pressing it (FIG. 13). As the materials used to fabricate TFQS are lightweight and thin, such devices can be easily integrated into any conventional packaging for various food items that can be monitored, such as via moisture content/humidity monitoring.

The working principle of the TFQS is depicted in FIG. 7b. Based on the data above, when its output was between 10 V and 20 V, it implied that the hardness of the crackers was acceptable (i.e., the “green light” on the GUI in FIG. 7c). Output between 21 V and 30 V, on the other hand, indicated that the hardness of the crackers was degrading and that they should be consumed soon. Lastly, output >50 V (indicating 75% RH or higher) or <10 V (indicating 99% RH) meant that consumers should refrain from eating the crackers as they may have gone bad (i.e., the “red light” on the GUI in FIG. 7c).

It is important to bear in mind that, although crackers' hardness might be partially restored by moving them from higher-RH to lower-RH storage levels, some negative changes in the quality attributes of low-moisture foods are non-recoverable and irreversible. Thus, rather than expecting consumers to rely solely on information provided via the proposed sensor hardware, public-information campaigns on the correct storage of low-moisture food could also usefully be designed.

In one or more embodiments, various concentrations of glycerol (or other materials used in a plasticizer, can be utilized to endow pectin film-based TENGs (or other carbohydrate-based films) with diverse characteristics. In the case of a relatively high glycerol concentration, i.e., PG-70 film, the resulting P-TENGs could convert mechanical energy into electricity more effectively than both their counterparts made from lower-glycerol PG-10, PG-30, and PG-50 film, and those made from higher-glycerol PG-90 film. When connected to appropriate power-management circuits, the optimal PG-70 film-based TENGs were found capable of charging capacitors and powering portable electronics such as a calculator and a hygrometer.

However, while the output of the PG-70 film-based TENGs increased significantly (p<0.05) as storage RH levels increased from 16% RH to 75% RH during sorption, sharply different results were obtained during desorption. On the other hand, the output of PG-50 film-based TENGs not only increased significantly (p<0.05) when storage RH levels increased; it also, unlike that of their PG-70-based counterparts, was close enough during sorption and desorption to be grouped, thus providing practically relevant humidity information.

As the quality of low-moisture food can be profoundly damaged by inappropriate RH levels during storage, the example further evaluated the quality attributes of an exemplar low-moisture food—crackers—focusing on their moisture content and hardness when stored at five different RH levels, as well as during sorption and desorption. In general, as final storage RH levels increased, the moisture content of the product increased and its hardness decreased. The data for these two quality attributes may imply the occurrence of hysteresis. This can be associated with changes in moisture content history. Nevertheless, the voltage data obtained during sorption and desorption can be reasonably grouped as an index of product quality.

Based on these data, a TFQS in this example was developed mainly of a PG-50 P-TENG incorporated it into a cracker box and connected to a GUI for displaying the results. Experiments with the TFQS revealed that it could successfully assess the quality of crackers at the moment of measurement. Of course, the quality of various other low-moisture foods is likely to be affected differently by storage conditions, but the embodiments of triboelectric sensors can be modified (e.g., as to concentrations, type of carbohydrate polymer, type of plasticizer, and so forth) to monitor their quality as well. In one embodiment, this might be achieved by 1) characterizing changes in quality attributes of different products, 2) adjusting PG films' glycerol concentrations, and/or 3) matching data obtained from triboelectric sensors to food-quality attributes.

It is of great importance to provide correct, readily understandable information about food quality changes to consumers and other stakeholders. For example, in the United States, about 20% of consumer-level food waste—valued at $32.2 billion annually—comprises the unnecessary disposal of wholesome food, primarily due to confusion about the meanings of date labels such as “Best if used by” vs. “Use by”. Providing scientifically accurate dynamic information about food quality can therefore empower consumers to make informed decisions about when to keep, consume, or discard food products, thus potentially alleviating the heavy economic, environmental, and societal burdens that food waste causes.

In sum, tuning P-TENGs' glycerol concentrations can dramatically boost their versatility. Additionally, it has highlighted the importance of evaluating triboelectric moisture-monitoring sensors by assessing their output not only during sorption, but also during desorption. The embodiments herein provide for enhancement to maintaining quality of low-moisture food, as well as empowering consumers to store their food products more effectively, thereby reducing food waste and rendering the food system more robust and sustainable.

Humidity can significantly impact the quality of food. In the case of low-moisture foods—a category characterized by its low water activity—humidity can cause undesirable physical and chemical changes. Intelligent packaging can be created for such foods based around versatile triboelectric nanogenerators (TENGs) that incorporate triboelectric layers fabricated from a carbohydrate polymer, such as pectin, and a plasticizer such as glycerol. Such TENGs can generate their highest electrical output, making them suitable for use as energy harvesters, when the glycerol content of such a layer was 70% of its pectin content. Such energy harvesters can successfully convert mechanical energy into sufficient electricity to power small electronic devices such as a sports watch and calculator. However, when the pectin-containing triboelectric layer's glycerol content was reduced to 50% of its pectin content, the resulting TENG-based sensors exhibited distinctive behaviors during sorption and desorption processes. Those behaviors were leveraged to create a triboelectric food-quality sensor (TFQS) that was integrated into food packaging for real-time food-quality monitoring. Testing of the TFQS indicated that it could effectively measure a key quality attribute, hardness, of our target low-moisture food, crackers. It should be understood that other carbohydrate-based films, other plasticizers, other concentrations of these materials (including other concentrations of pectin and glycerol) can also be utilized.

These findings illustrate not only how altering their compositions can endow triboelectric devices with multifunctionality, but also such devices' potential to help reduce food waste by providing consumers with accurate, dynamic quality information, addressing a core limitation of the current pre-printed food-date label system, which does not account for storage conditions.

In other embodiments, the organic-based (such as including pectin films) devices can be utilized as a TENG for harvesting ambient mechanical energy from our everyday life (for example, walking or running). A typical human motion is below 4 Hz but the TENG can convert this mechanical energy into electrical energy.

In one embodiment, the P-TENG can include Layer 1: Pectin/glycerol, copper, acrylic; and Layer 2: PTFE, copper, acrylic. In another embodiment, the P-TENG can include Layer 1: pectin/glycerol, ITO/PET (where ITO serves as a conductor, and PET as a substrate; and Layer 2: PTFE, ITO/PET.

In one or more embodiments, 70% glycerol could also be used for other types of low-moisture foods; in other embodiments various ranges can be utilized such as a range of 10% to 70%. In one embodiment, water can be utilized or added to dissolve the pectin powder, and it (or most of it) may be evaporated during the film-making process.

In one embodiment, the triboelectric devices include or consist of two main layers: The first layer includes pectin/glycerol, copper, and acrylic (as a substrate). The second layer includes PTFE, copper, and acrylic (as a substrate). The substrate can be various materials including paper.

In one embodiment, mechanical energy generated from handling the food packaging can include tapping or other force applied to the packaging, movement of the packaging, and so forth.

In one embodiment, the P-TENG can be operated using a “contact-separation mode” (e.g., layers physically brought into contact via application of external forces, then separated by releasing those forces). It should be understood that other materials, other arrangements, and/or other organic material can be utilized herein which may or may not be utilized with pectin.

FIG. 8 illustrates COMSOL simulations of the electrical potential of a pectin-glycerol film-based triboelectric nanogenerator with the distance between the two triboelectric layers being I. 1 mm and II. 10 mm. FIG. 9 illustrates outputs of pectin-glycerol film-based triboelectric nanogenerators made from films with five different proportions of glycerol. FIG. 10 illustrates output stability of triboelectric nanogenerators made from pectin-glycerol films whose glycerol content weighs 70% as much as their pectin content (i.e., PG-70 films). FIG. 11 illustrates voltage curves of triboelectric nanogenerators made from pectin-glycerol films whose glycerol content weighs 70% as much as their pectin content (i.e., PG-70 films). FIG. 12 illustrates a variation in the dry-basis moisture content of PG-50 film (i.e., pectin-glycerol film whose glycerol content weighed 50% of its pectin content) when stored at 16% relative humidity over 72 h. FIG. 13 illustrates digital photographs showing changes in the appearance of a piece of semi-transparent PG-50 film (i.e., pectin-glycerol film whose glycerol content weighed 50% of its pectin content) that was attached to a piece of copper film and stored at 16% relative humidity (RH) for 72 h, then at 99% RH for 72 h. FIG. 14 illustrates voltage outputs of triboelectric nanogenerators made from PG-70 film (i.e., pectin-glycerol film whose glycerol content weighed 70% of its pectin content) stored at five different relative-humidity levels. Asterisks indicate that, at a given relative-humidity level, there was a significant difference between the voltage outputs after the water-sorption and water-desorption processes (p<0.05).

While for purposes of simplicity of explanation, the respective processes described herein may include a series of steps, it is to be understood and appreciated that the claimed subject matter may utilize another order of the steps, as some blocks may occur in different orders and/or concurrently with other steps from what is depicted and described herein. Moreover, not all illustrated steps may be required to implement the methods described herein.

Any use of the terms “first,” “second,” and so forth, in the claims, unless otherwise clear by context, is for clarity only and doesn't otherwise indicate or imply any order in time. For instance, “a first determination,” “a second determination,” and “a third determination” does not indicate or imply that the first determination is to be made before the second determination, or vice versa, etc.

While various components have been illustrated as separate components, it will be appreciated that multiple components can be implemented as a single component, or a single component can be implemented as multiple components, without departing from example embodiments.

Unless otherwise explained, all technical and scientific terms used herein have the same meaning as commonly understood by one of ordinary skill in the art to which a disclosed disclosure belongs. In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

As used herein, “comprising” is synonymous with “including,” “containing,” or “characterized by,” and is inclusive or open-ended and does not exclude additional, unrecited elements or method steps. For instance, “comprising A or B” means “including A” or “including B” or “including A and B.” As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein suitably may be practiced in the absence of any element or elements, limitation or limitations which is not specifically disclosed herein.

As may also be used herein, the term(s) “operably coupled to,” “coupled to,” and/or “coupling” includes direct coupling between items and/or indirect coupling between items via one or more intervening items. Such items and intervening items include, but are not limited to, junctions, communication paths, components, circuit elements, circuits, functional blocks, and/or devices. As an example of indirect coupling, a signal conveyed from a first item to a second item may be modified by one or more intervening items by modifying the form, nature or format of information in a signal, while one or more elements of the information in the signal are nevertheless conveyed in a manner than can be recognized by the second item. In a further example of indirect coupling, an action in a first item can cause a reaction on the second item, as a result of actions and/or reactions in one or more intervening items.

The disclosure may be further understood by the included non-limiting examples. All references cited herein are hereby incorporated by reference to the extent not inconsistent with the disclosure herewith.

Although the description herein contains many specificities, these should not be construed as limiting the scope of the disclosure, but as merely providing illustrations of some of the presently preferred embodiments of the disclosure. Thus, the scope of the disclosure should be determined by the appended aspects and their equivalents, rather than by the examples given.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure. Thus, it should be understood that, although the present disclosure has been specifically disclosed by preferred embodiments, exemplary embodiments, and optional features, modification and variation of the concepts herein disclosed may be resorted to by those skilled in the art, and that such modifications and variations are considered to be within the scope of this disclosure as defined by the appended aspects. The specific embodiments provided herein are examples of useful embodiments of the present disclosure and it will be apparent to one skilled in the art that the present disclosure may be carried out using a large number of variations of the devices, device components, and methods steps set forth in the present description. As will be obvious to one of skill in the art, methods and devices useful for the present methods can include a large number of optional composition and processing elements and steps. Accordingly, the embodiments disclosed and/or claimed herein are intended to embrace all such modifications and variations that fall within the spirit and scope of the appended claims.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein.

Although the present disclosure has been described with reference to certain embodiments thereof, other embodiments are possible without departing from the present disclosure. The spirit and scope of the appended aspects should not be limited, therefore, to the description of the preferred embodiments contained herein. All embodiments that come within the meaning of the aspects, either literally or by equivalence, are intended to be embraced therein. Furthermore, the advantages described above are not necessarily the only advantages of the disclosure, and it is not necessarily expected that all of the described advantages will be achieved with every embodiment of the disclosure.

Although specific embodiments have been illustrated and described herein, it should be appreciated that any arrangement which achieves the same or similar purpose may be substituted for the embodiments described or shown by the subject disclosure. The subject disclosure is intended to cover any and all adaptations or variations of various embodiments. Combinations of the above embodiments, and other embodiments not specifically described herein, can be used in the subject disclosure. For instance, one or more features from one or more embodiments can be combined with one or more features of one or more other embodiments. In one or more embodiments, features that are positively recited can also be negatively recited and excluded from the embodiment with or without replacement by another structural and/or functional feature. The steps or functions described with respect to the embodiments of the subject disclosure can be performed in any order. The steps or functions described with respect to the embodiments of the subject disclosure can be performed alone or in combination with other steps or functions of the subject disclosure, as well as from other embodiments or from other steps that have not been described in the subject disclosure. Further, more than or less than all of the features described with respect to an embodiment can also be utilized.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative (rather than in a restrictive) sense.

In addition, a flow diagram may include a “start” and/or “continue” indication. The “start” and “continue” indications reflect that the steps presented can optionally be incorporated in or otherwise used in conjunction with other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The Abstract of the Disclosure is provided with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

Various other components and functions (whether alone, in place of, or in combination with the components and functions described herein) can be used in energy harvesting and/or self-powered sensing devices utilizing natural polymeric materials, such as for triboelectric nanogenerator.

In one or more embodiments, the triboelectric sensor is integrated into food packaging to provide real-time monitoring, wherein the triboelectric sensor comprises one of a first sensor having a first layer including pectin, glycerol, copper, and acrylic and having a second layer including PTFE (or Kapton), copper, and acrylic or a second sensor having a first layer including pectin, glycerol, ITO and PET and having a second layer including PTFE (or Kapton), ITO and PET.

What is claimed is:

1. A food monitoring device comprising:
a triboelectric sensor including a film comprising a carbohydrate polymer and a plasticizer, wherein the carbohydrate polymer includes pectin and the plasticizer includes glycerol, wherein a concentration of the glycerol is approximately 50% by weight relative to the pectin, and wherein the triboelectric sensor is configured to monitor food according to an electrical signal that is generated and which varies based on histories of moisture content change, thereby providing real-time monitoring of food quality.

2. The food monitoring device of claim 1, wherein the triboelectric sensor is integrated into food packaging to provide real-time monitoring, wherein the triboelectric sensor comprises one of a first sensor having a first layer including pectin, glycerol, copper, and acrylic and having a second layer including polytetrafluoroethylene (PTFE) or Kapton, copper, and acrylic or a second sensor having a first layer including pectin, glycerol, indium tin oxide (ITO) and polyethylene terephthalate (PET) and having a second layer including PTFE or Kapton, ITO and PET.

3. The food monitoring device of claim 1, further comprising a graphical user interface (GUI) configured to display real-time data based on the real-time monitoring.

4. The food monitoring device of claim 3, wherein the GUI is configured to alert users when food quality according to the generated electrical signal falls below or above predetermined thresholds.

5. The food monitoring device of claim 1, wherein the triboelectric sensor is powered solely by mechanical energy generated from handling the food packaging, and wherein the triboelectric sensor is incorporated into a box serving as the food packaging.

6. A triboelectric device comprising:
a device body including a carbohydrate polymer, a plasticizer, and multiple layers of material, wherein the carbohydrate polymer includes pectin and the plasticizer includes glycerol, wherein a concentration of the glycerol is approximately 50% by weight relative to the pectin if the triboelectric device is being used as a sensor or is approximately 70% by weight relative to the pectin if the triboelectric device is being used as an energy harvester, wherein electrical energy is generated when mechanical energy is applied to the device body.

7. The triboelectric device of claim 6, wherein the sensor is configured to monitor food according to an electrical signal that is generated and which varies based on histories of moisture content change, thereby providing real-time monitoring of food quality, and wherein the multiple layers of material comprise two main layers each including an active layer, a conductor, and a substrate.

8. The triboelectric device of claim 7, wherein the material includes copper, acrylic, PTFE/Kapton, ITO and PET.

9. The triboelectric device of claim 7, wherein the mechanical energy comprises a vertical force applied to the device body and/or a lateral force applied to the device body.

10. The triboelectric device of claim 7, wherein the device body is integrated into food packaging to provide real-time monitoring.

11. The triboelectric device of claim 7, further comprising a graphical user interface (GUI) configured to display real-time data based on the real-time monitoring.

12. The triboelectric device of claim 11, wherein the GUI is configured to alert users when food quality according to the generated electrical signal falls above or below predetermined thresholds.

13. A method comprising:
determining a concentration for a carbohydrate polymer and a plasticizer to be used in a triboelectric device comprising multiple layers of material, wherein the triboelectric device is to be used as one of a sensor or an energy harvester, wherein the concentration is different depending on usage as the sensor or the energy harvester, and wherein the determining the concentration is based on improving operation as the sensor or improving operations as the energy harvester;
providing the triboelectric device including the polymer and the plasticizer with an object that is subjected to mechanical energy; and
connecting the triboelectric device to an electrical component, wherein when the mechanical energy is applied to the device body electrical energy is generated and provided to the electrical component.

14. The method of claim 13, wherein the carbohydrate polymer includes pectin and the plasticizer includes glycerol, wherein the concentration of glycerol is approximately 50% by weight relative to the pectin if the triboelectric device is being used as the sensor or is approximately 70% by weight relative to the pectin if the triboelectric device is being used as the energy harvester.

15. The method of claim 13, wherein the object is food packaging, wherein the triboelectric device is configured to monitor food according to an electrical signal that is generated and which varies based on histories of moisture content change, thereby providing real-time monitoring of food quality, wherein the object is a food item, and further comprising:
determining food quality data for the food item according to relative humidity exposure histories for the food item, wherein the real-time monitoring is based on the food quality data.

16. The method of claim 13, wherein the material includes copper, acrylic, PTFE/Kapton, ITO and PET.

17. The method of claim 13, wherein the mechanical energy comprises a vertical force and/or a lateral force applied to the triboelectric device through the object.

18. The method of claim 13, wherein the triboelectric device is integrated into food packaging to provide real-time monitoring.

19. The method of claim 13, further comprising displaying real-time data via a graphical user interface (GUI) based on real-time monitoring provide via the sensor.

20. The method of claim 19, wherein the GUI is configured to alert users when food quality according to the real-time monitoring falls above or below predetermined thresholds.
